(19) United States
(12) Patent Application Publication
(10) Pub. No.: US 2023/0381782 A1
(43) Pub. Date: Nov. 30, 2023

(54) SEQUENCING SYSTEMS AND METHODS UTILIZING NON-PLANAR SUBSTRATES

(71) Applicant: MGI Tech Co., Ltd., Shenzhen (CN)

(72) Inventors: Paul Lundquist, Oakland, CA (US); Joon Yang, San Jose, CA (US); Jon Bartman, San Jose, CA (US); Chintang Yen, San Jose, CA (US); Razvan Chirita, San Jose, CA (US); Kee Tsz Woo, San Jose, CA (US); Jay Shafto, San Jose, CA (US); Michelle Jarrell, San Jose, CA (US); Wei Wang, San Jose, CA (US)

(21) Appl. No.: 18/249,563

(22) PCT Filed: Nov. 2, 2021
(86) PCT No.: PCT/CN2021/128020

§ 371 Date: Apr. 19, 2023

Related U.S. Application Data
(60) Provisional application No. 63/108,818, filed on Nov. 2, 2020.

(51) Int. Cl. B01L 7/00

(52) U.S. Cl. CPC B01L 7/52; B01L 2300/0841; B01L 2300/0636

(57) ABSTRACT

A nucleic acid sequencing system may include non-planar substrates coupled to the outer surface of a rotating drum. The substrates may be curved and include a plurality of nucleic acid samples. A detection system, including for example an objective and a camera, may detect sequencing events on the non-planar substrate while the non-planar substrate is rotated relative to the detection system around a longitudinal axis of the drum by the actuation system.

### RELATED FIELDS

This disclosure relates to systems for nucleic acid sequencing and other biochemical analyses.

### BACKGROUND

Nucleic acid sequencing includes numerous different costs, for example, costs related to the purchase and upkeep of the sequencing device. Reducing the amount of time to produce the same amount of sequencing data compared to existing sequencing devices may reduce the overall costs of producing the sequencing data.

Some currently available sequencing systems rely detect sequencing events on an essentially 2-dimensional planar substrate of a flowcell. An objective of an optical detection system and the flowcell are moved relative to each other so that the field of view of the objective is passed over the substrate a plurality of times, wherein each pass images a portion of the substrate so that the entire substrate is imaged. These systems have the disadvantages of needing to slow, stop, and/or change the direction of the relative movement of the objective of the optical system relative to the substrate between the multiple transits over a flowcell needed to image the entire substrate of the flowcell. This leads to periods of time during the overall imaging process during which imaging of the substrate is not taking place due to the need to position and control the relative movement of the system components in order to resume imaging. Accordingly, there is a need to reduce or eliminate this downtime.

### BRIEF SUMMARY

This disclosure presents systems and methods for detecting sequencing events. The systems and methods may be employed in, for example, sequencing nucleic acid molecules disposed on a substrate, wherein the substrate may include from millions to billions of individual nucleic acid sites. The substrate may be formed or coupled to an outer cylindrical surface of a drum so that the substrate is curved. The drum may rotate relative to a field of view (FOV) of a detection system, for example an objective of an optical detection system, so that the FOV passes over the curved substrate in order to image the sequencing events on the curved substrate. One advantage of the disclosed systems and methods for detecting sequencing events may be improved throughput due to increasing the distance of the substrate that the FOV of the imaging system can cover while continuously imaging the sequencing events on the substrate without slowing or stopping relative movement between the FOV and the substrate, thereby creating significant cost savings as will be discussed herein.

In some embodiments, the technology may include nucleic acid sequencing system. Nucleic acid sequencing system may include a drum defining an outer surface and a longitudinal axis. Nucleic acid sequencing systems may further include a non-planar substrate coupled to the outer surface of the drum and designed to support a plurality of nucleic acid samples. Nucleic acid sequencing systems may further include an actuation system designed to rotate the drum around the longitudinal axis. Nucleic acid sequencing system may further include a detection system designed to detect sequencing events on the non-planar substrate while the non-planar substrate is rotated relative to the detection system around the longitudinal axis by the actuation system.

In some embodiments, the outer surface of the drum may be cylindrical. In some embodiments, the non-planar substrate may be curved around the outer surface of the drum. In some embodiments, the actuation system may be designed to translate the drum along the longitudinal axis, and the detection system may be designed to detect sequencing events on the non-planar substrate while the non-planar substrate is translated relative to the detection system along the longitudinal axis by the actuation system. In some embodiments, the detection system may be an optical detection system including at least one objective, for example one, two, three or more objectives. In some embodiments, the at least one objective may include two objectives for imaging different portions of the substrate, including portions offset radially and longitudinally of the longitudinal axis of the drum.

In some embodiments, a nucleic acid sequencing system may additionally include a drum assembly. A drum assembly may include the drum, and an outer drum shell defining an interior cavity. The inner drum may be positioned within the inner cavity, and the actuation system may rotate the inner drum within the inner cavity of the outer drum shell. In some embodiments, a nucleic acid sequencing system may also include a track assembly coupled to the drum assembly. The actuation system may translate the drum assembly in a direction parallel to the longitudinal axis in order for the at least one objective to image different portions of the curved substrate in a direction parallel to the longitudinal axis as the inner drum is rotating around the longitudinal axis. In some embodiments, a nucleic acid sequencing system may also include a control system. The control system may control the actuation system in order to rotate the inner drum and translate the inner drum in order for the objective to image a predefined imaging path on the curved substrate. In some embodiments, the predefined imaging path includes a ring around a circumference of the inner drum. In some embodiments, the predefined imaging path includes a spiral winding around the inner drum a plurality of times.

In some embodiments, the drum includes a plurality of ridges, and a plurality of recessed surface between adjacent ridges of the plurality of ridges comprising a first recessed surface. In some embodiments, the non-planar substrate is coupled to the first recessed surface.

In some embodiments, a nucleic acid sequencing system may include a fluid delivery system to deliver fluid to the interior cavity of the outer drum shell in order to perform a sequencing process on the non-planar substrate. The fluid delivery system may include a jetting print head to jet droplets of a reagent onto the non-planar substrate. An outer drum shell may include an exit port to drain fluid within the interior cavity delivered by the fluid delivery system. A fluid delivery system may include a recycling system for capturing fluid drained from the exit port in order to reuse the fluid.

In some embodiments, a non-planar substrate may include an ordered array of discrete spaced apart regions (“spots”). The discrete spaced apart regions may be adapted to immobilize nucleic acids. In some embodiments, a nucleic acid sequencing system may include nucleic acids immobilized on the discrete spaced apart regions of the array. The nucleic acids immobilized on the discrete spaced apart regions may be DNBs or PCR products.

In some embodiments, the technology relates to a method of nucleic acid sequencing. The method may include rotating a drum defining an outer surface around a longitudinal axis of the drum with an actuation system. The method may also include detecting sequencing events, with a detection system, on a non-planar substrate coupled to the drum while the non-planar substrate is rotated relative to the detection system around the longitudinal axis by the actuation system. Detecting sequencing events may be performed while the drum is rotated at a constant speed. Detecting sequencing events on the non-planar substrate may include positioning an objective of the detection system at a first longitudinal position relative to the longitudinal axis of the drum, maintaining the objective at the first longitudinal position as the drum is rotated relative to the detection system around the longitudinal axis by the actuation system at least one full rotation in order to image a first portion of the non-planar substrate around a first ring imaging path, positioning the objective at a second longitudinal position relative to the longitudinal axis of the drum; and maintaining the objective at the second longitudinal position as the drum is rotated relative to the detection system around the longitudinal axis by the actuation system at least one full rotation in order to image a second portion of the non-planar substrate, different than the first portion, around a second ring imaging path. Detecting sequencing events on the non-planar substrate may include positioning an objective of the detection system at a first longitudinal position relative to the longitudinal axis of the drum, and translating the objective at a constant speed from the first longitudinal position to a second longitudinal position as the drum is rotated relative to the detection system around the longitudinal axis by the actuation system in order to image a spiral imaging path around the non-planar substrate.

### BRIEF DESCRIPTION OF THE DRAWINGS

FIGS. 1A-1E show embodiments of an optical imaging system.

FIGS. 2A-2D show relative movements of an objective relative to an inner drum.

FIGS. 3A-3D show curved substrates with ring imaging paths around an inner drum.

FIGS. 4A-4C show curved substrates with spiral imaging paths around an inner drum.

FIGS. 5A-5C show embodiments of an optical imaging system with multiple objectives.

FIGS. 6A and 6B show embodiments of reagent delivery systems.

FIGS. 7A-7C show embodiments of jetting reagent delivery systems.

FIG. 8 shows an embodiment of a process diagram.

FIG. 9 shows a control system schematic.

In accordance with common practice, the described features and elements are not drawn to scale but are drawn to emphasize features and elements relevant to the present disclosure.

### DETAILED DESCRIPTION

The present disclosure describes a sequencing detection system that may be employed in detecting sequencing events on a curved substrate. For example, the disclosed sequencing detection system may be an optical imaging system employed in sequencing for example, nucleic acids. In embodiments, the template nucleic acid molecules may be bound to, or otherwise disposed on, a surface of the curved substrate and then imaged by the optical imaging system.

There are many approaches to nucleic acid (e.g., DNA) sequencing. See, e.g., Kumar, K., 2019, “Next-Generation Sequencing and Emerging Technologies,” Semin Thromb Hemost 45(07): 661-673. The most popular methods use arrays with a large number of discrete sites (e.g., 100 million to 1 billion or more) in an ordered array on a planar substrate. Typically the sites are small (e.g., characterized by a diameter or diagonal less than 1 micrometer, often less than 500 nanometers, and often in the range of 50 nanometers to 500 nanometers) and present at a high density (e.g., of more than ˜10⁶ sites per cm²). Nucleic acid templates are immobilized directly or indirectly at the individual sites for sequencing. Generally each site contains a clonal population of template sequences, such as a DNA nanoball (Complete Genomics, Inc.) or PCR products or amplicons (Illumina, Inc.). For illustration and not limitation, in these approaches nucleic acid sequences are determined one base at a time over a series of sequencing “cycles.” Each cycle comprises (i) introducing reagents to each site on the array of immobilized template molecules; (ii) carrying out a series of biochemical or enzymatic reactions (“sequencing reactions”) simultaneously at the sites; (iii) detecting signals at each site (zero, one or more than one signal per site per cycle) which may be referred to as “image acquisition:”; and (iv) carrying out enzymatic, washing, or regeneration steps at each site on the array so that another sequencing cycle can be carried out. Without limitation the “signals” collected in (iii) may be optical signals, e.g., fluorescence or luminescence signals. The sequencing array is usually contained in a “flow cell” through which primers, reagents, washes, etc. can be flowed. Typically a sequencing run consists of ˜400 cycles, which means that ˜400 or more imaging events, each involving acquiring signal individually from each of millions of sites is required. The speed and precision of image collection affects cost, efficiency, and sequencing data quality.

As used herein a “sequencing event” refers to emission of an optical signal (e.g., a fluorescence or luminescence signal) resulting from a sequencing process. An exemplary sequencing process is a cycle of a sequencing-by-synthesis process. In this approach, nucleotides are incorporated into a primer extension product (e.g. using reversible terminator nucleotides). In this approach, nucleotides can be labeled with, for example, a fluorescent dye or a source of a luminescence signal (e.g. luciferase or luciferase substrate). A luminescent signal includes chemiluminescence and bioluminescence. A nucleotide can be labeled directly with a fluorescent dye or a source of a luminescence signal or can be associated with an antibody, aptamer or other agent labeled with a signal generating moiety. In the process of sequencing a defined optical signal is produced at each site in an array by, for example, illumination of the fluorescent dye(s) with an excitation wavelength, and the signals and corresponding positions are recorded.

Although framed in the context of nucleic acid sequencing, it will be recognized that the devices and methods disclosed herein are not limited to nucleic acid sequencing uses. The system may be used, for example, for nucleic acid analysis other than sequencing (e.g., SNP analysis, real time PCR analysis) or for analysis of chemical or biochemical processes using substrates or analytes other than nucleic acids. In one aspect the invention provides an assay system comprising a drum defining an outer surface and a longitudinal axis non-planar substrate coupled to the outer surface of the drum and configured to support a plurality of chemical or biochemical reactions, an actuation system configured to rotate the drum around the longitudinal axis; and a detection system configured to detect optical signals produced by the chemical or biochemical reactions on the non-planar substrate while the non-planar substrate is rotated relative to the detection system around the longitudinal axis by the actuation system.

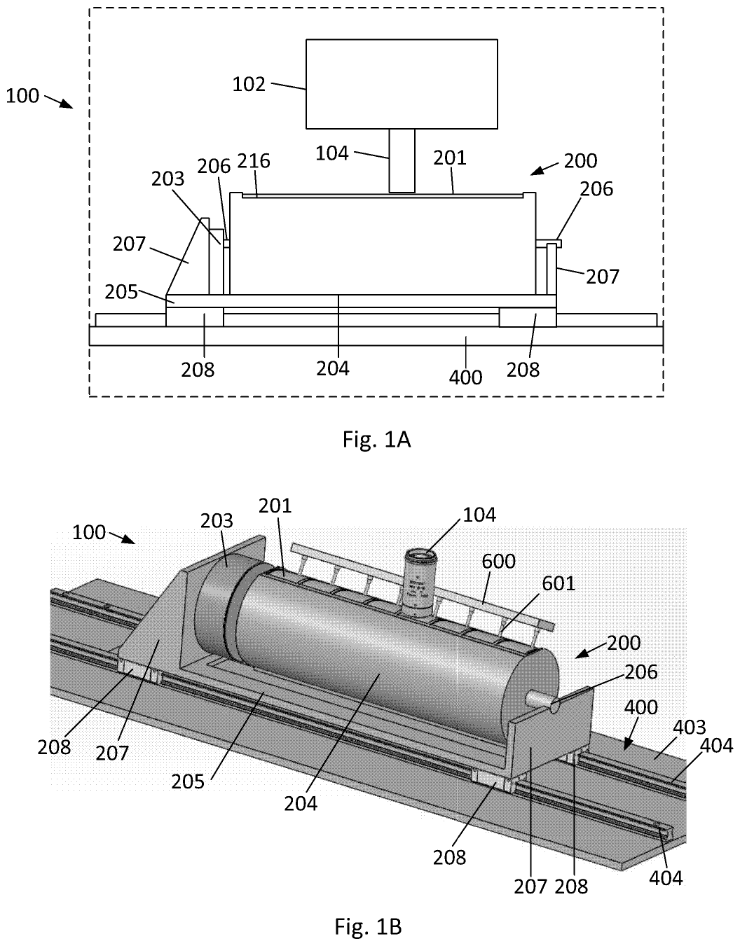

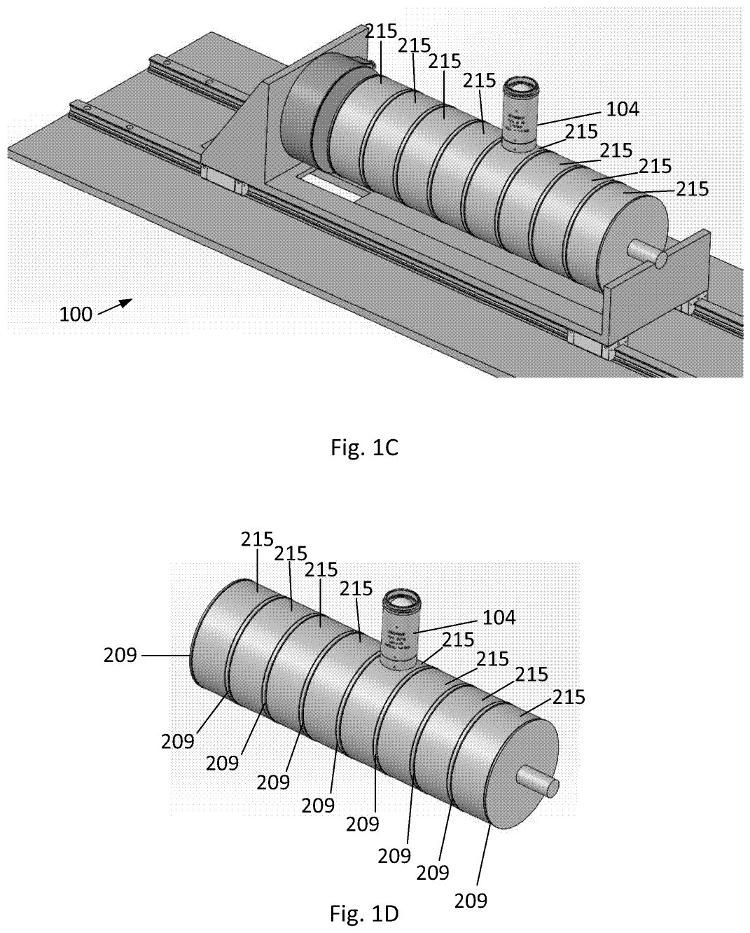

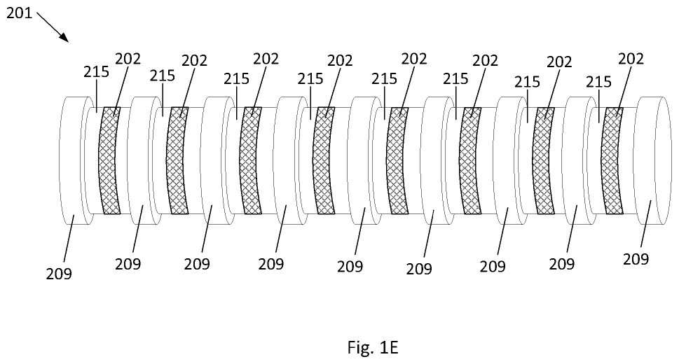

FIGS. 1A-1C show examples of a sequencing detection system 100 according to the present technology. FIG. 1A shows a schematic view of a sequencing detection system 100 comprising a detection system 102, in the form of an optical detection system including an objective 104, a drum assembly 200, a fluid delivery system 600, and a track assembly 400. The detection system 102 may be an optical detection system further including camera(s), processor(s), lens(es), illumination source(s), filter(s), mirror(s), and actuator(s) used for detecting sequencing events on a substrate. Examples of detection systems include one or more of objective lens, laser illumination systems, autofocus systems, systems of dichroic filters to combine illumination and detection paths and to provide paths for autofocus, and high sensitivity cameras. Cameras may be, for example, in area scan or Time-Domain-Integration (TDI) formats. For example, the detection system 102 may include a Time Delay Integration (TDI) camera with a sensor specified for 8900×256 pixels at a 500 kHz line rate.

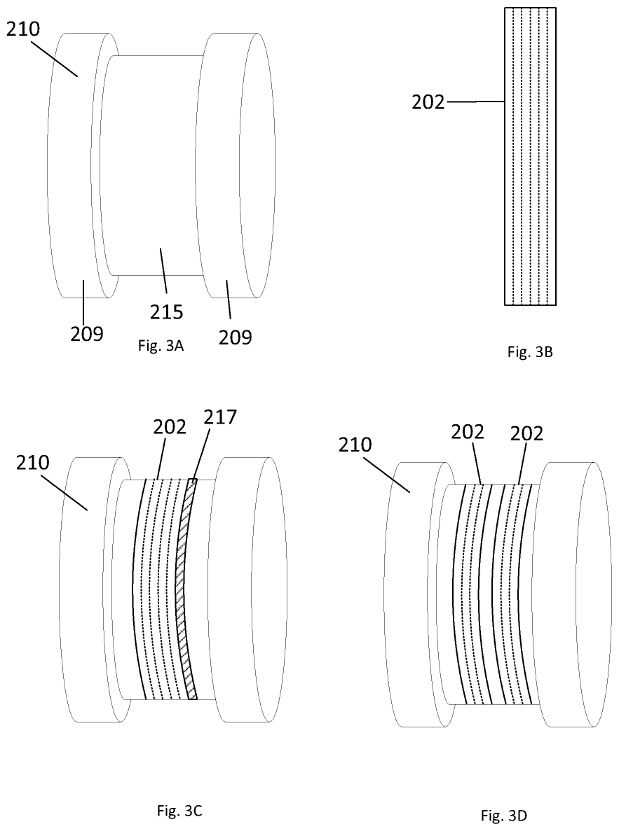

As shown for example in FIG. 1B, the drum assembly 200 comprises an inner drum 201. As will be described in greater detail below, the inner drum 201 may be generally cylindrical and comprises one or more substrates 202, for example as shown in FIG. 1E, on an outer surface of the inner drum 201. The outer surface of the inner drum 201 may be cylindrical, and the substrates 202 may be curved to match the radius of a cylindrical inner drum 201, for example as shown in FIG. 3C. The drum assembly 200 further comprises a rotational actuator 203, for example a motor, as part of an actuation system of the sequencing detection system 100. The rotational actuator 203 is used for rotating the inner drum 201, and therefore the substrate 202, relative to the objective 104 in order to image different portions of the substrate 202. Further, the actuation system of the sequencing detection system 100 may include additional actuators configured to cause relative motion between the objective and the curved substrate in multiple degrees of freedom, for example any combination of translations in up to three orthogonal directions and/or rotations around up to three orthogonal axes.

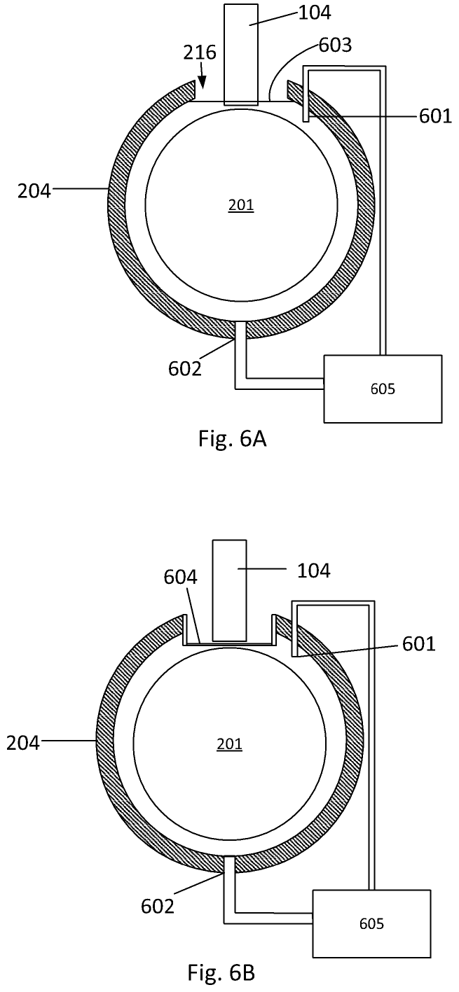

As shown for example in FIG. 1B, the drum assembly 200 in addition to the inner drum 201, includes an outer drum shell 204 rotationally coupled to and positioned around the inner drum 201, and a platform 205 fixedly coupled to the outer drum shell 204. As shown in FIG. 1B, the outer drum shell 204 may be substantially cylindrical in shape and defines an interior cavity, which may be substantially cylindrical. The interior cavity may be shaped and sized to correspond to the shape and size of the inner drum 201. The inner drum 201 is positioned within the interior cavity of the outer drum shell, as shown for example in FIG. 6A. The outer drum shell 204 may define one or more openings 216 providing optical access for the objective 104 to image the substrate 202 and/or providing fluid delivery access to a volume between the inner drum 201 and the inner surface of the outer drum shell 204. During imaging, the end of the objective 104 may be positioned within the interior cavity of the outer drum shell 204. The opening 216 may be uncovered so that a fluid surface of fluids within the interior cavity between the inner drum 201 and outer drum shell 204 are open to the environment. The environment around the opening 216 may be controlled in at least one of temperature, humidity, and elemental atmosphere composition. The outer drum shell 204 may be fixedly coupled to the platform 205 so that the inner drum 201 may be rotated relative to both the platform 205 and the outer drum shell 204.

FIG. 1C shows the sequencing detection system 100 of FIG. 1A with the outer drum shell 204 omitted for clarity purposes. As shown for example in FIG. 1C, the inner drum 201 may be substantially cylindrical. The inner drum may be formed of metal and/or polymer. For example, the inner drum may be made of one or more of aluminum, steel, Ultem, and polycarbonate. The inner drum 201 may be molded (e.g. injection molded) and/or machined (e.g. with a CNC lathe). The inner drum 201 may have an outer diameter between 10 mm and 1000 mm. The inner drum 201 comprises axles 206 extending through ends of the outer drum shell 204 and rotatably supported by brackets 207 on the platform 203. The brackets may include bearings supporting the axles 206 so that the inner drum may rotate relative to the platform, and so that the inner drum 201 is restrained relative to the platform in all but a single rotational degree of freedom. The actuator 203 of the drum assembly 200 may be coupled to the axle 206 and may be for example a stepper motor, a servo motor, or the like, in order to cause rotation of the inner drum 201 relative to the outer drum shell 204, the platform 203, and the objective 104 of the detection system. The actuator 203 may include a feedback loop and/or a flywheel in order to maintain a constant rotational speed. The inner drum may be rotated for example between 5 RPM and 1000 RPM during imaging of the substrate. The rotational speed of the inner drum may be selected based on a camera frame rate and a magnification of the optical system in combination with the diameter of the inner drum. TDI cameras may have a frame rates between 50,000 lines/sec and 1,000,000 lines/sec. For example, a camera may have a line rate of 250,000 lines/sec and a magnification of 18×, results in a linear speed up to 72 mm/sec. The rotational speed of the inner drum may then be selected so that the linear speed of the surface of the substrate on the inner drum moving past the FOV of the camera does not exceed the linear speed of the camera system. For example, an inner drum with a diameter of 100 mm will be selected to have a rotational speed of less than 0.23 rotations per second ((72 mm/sec)/(100 mm*pi/1 rev).

The track assembly 400 may comprise a base 403 and one or more tracks 404, for example two tracks as shown in FIGS. 1B and 1C. The platform 203 includes sliders 208 slidably coupled to the tracks 404 in order to allow translation of the drum assembly 200 in one direction while restraining motion in other directions relative to the objective 204. As used herein, translation in the direction of the track 404 will be referred to as translation in the X-direction, in an XYZ reference frame. As will be discussed in greater detail below, translation of the inner drum 201 along a longitudinal axis of the inner drum in the X-direction, and rotation of the inner drum 201 the longitudinal axis of the inner drum around the X-axis, is used to cause relative movement between the objective 104 and the substrate 202 in order to image sequencing events around a circumference and width of the substrate 202.

As shown in FIG. 1D, the inner drum 201 may include ridges 209 defining distinct portions of the inner drum 201 each including a recessed surface 215 between two ridges 209. The ridges 209 may be between 50 microns and 1.0 mm in height and between 50 microns and 1.0 mm in width. Sealing elements may be positioned between the inner surface of the interior cavity of the outer drum shell 204 and the ridges 209 of the inner drum 201 in order to define fluidically separated chambers for each of the distinct portions of the inner drum 201. The sealing members may include O-rings or gaskets. The O-rings or gaskets may be seated in grooves formed into the inner surface of the interior cavity of the outer drum shell 204. Each chamber may have a dedicated fluid delivery sub-system of the fluid delivery system 600 so that each chamber acts as a discrete flowcell wherein distinct reactions may simultaneously occur in the discrete flowcells. Each discrete flowcell may include one or more dedicated temperature control devices. Temperature control devices may include one or more of: a heating/cooling element controlling the temperature of the outer wall of the fluidic outer drum, an embed heating/cooling element inside of the inner drum, and a heating/cooling element controlling the temperature of fluids (e.g. the reagents) which are cycled in and out of each flowcell, for example by maintaining the temperature of individual reagents in reservoirs.

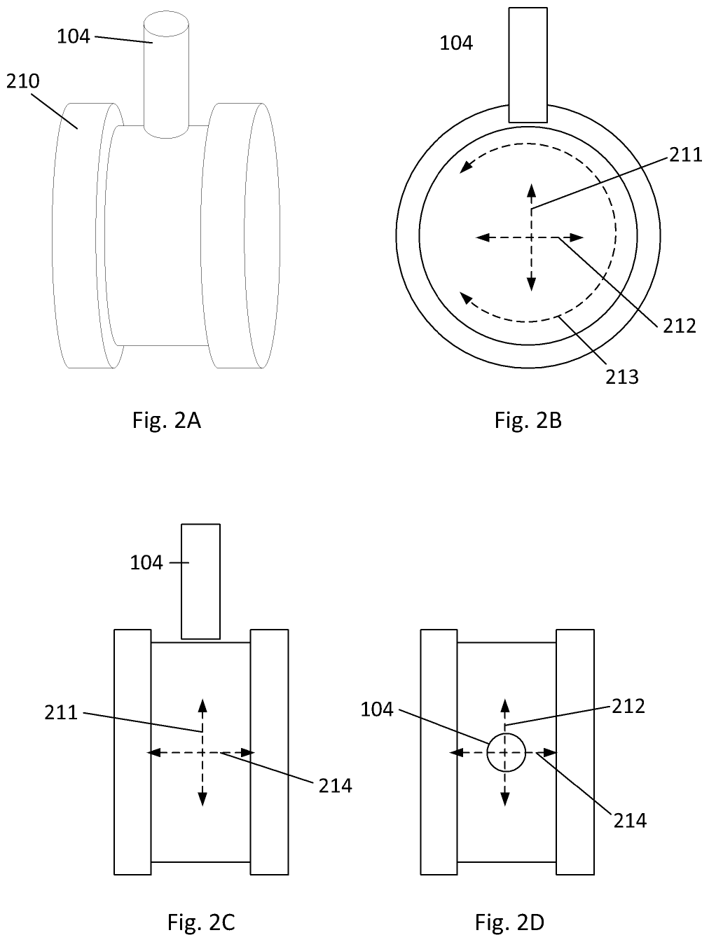

FIGS. 2A-2D show a portion 210 of an inner drum 201 and an objective 104, and further include indications of relative movement between the inner drum 201 and objective 104 which may be performed by actuators of an actuation system. The relative movement between the inner drum 201 and the objective 104 may be performed by actuators controlled by a control system in order to continuously maintain a tangential relationship between a rotating curved substrate 202 and the FOV of the objective 104 so that FOV is maintained in focus on the desired portion of the curved substrate. FIG. 2A shows a schematic of an example of a portion 210 of an inner drum 201 and an objective 104 positioned over the inner drum 201, and as noted the relative motion between the inner drum 201 and the objective 104 may be used to image different portions of a curved substrate 202 on the outer surface of the inner drum 201.

FIG. 2B shows an end view of a cross-section of a portion 210 of the inner drum 201 and the objective 104. As shown, the inner drum 201 may move relative to the objective 104 in a vertical Z direction 211 perpendicular to the longitudinal X-axis of the inner drum 201, and in a horizontal Y direction 212 perpendicular to the longitudinal X-axis of the inner drum 201 The relative translation movements shown in FIG. 2B may be achieved by translationally moving the drum assembly 200 relative to a stationary objective 104, translationally moving the objective 104 and optionally the detection system associated with the objective 204 relative to a translationally stationary drum assembly 200, or translationally moving both the objective 104 and drum assembly 200 relative to each other and a fixed frame of reference. Actuators for performing these translational movements may be coupled to one or more of the drum assembly 200, the track assembly 400, and the detection system 102 associated with the objective 104. FIG. 2B further shows the inner drum 201 being rotationally moveable in a rotational direction 213 around the X-axis, as discussed above relating to the actuator 203.

FIG. 2C shows a side view of the inner drum 201 and the objective 104. As shown, the inner drum 201 may move relative to the objective 104 in a vertical Z direction 211 perpendicular to the longitudinal X-axis of the inner drum 201 as noted above regarding FIG. 2B, and further in the horizontal X direction 214. In embodiments, the relative X direction 214 translation movements shown in FIG. 2C may be achieved by translationally moving the drum assembly 200 relative to a stationary objective 104, for example using the track system 400, translationally moving the objective 104 and optionally the detection system associated with the objective 204 relative to a translationally stationary drum assembly 200, or translationally moving both the objective 104 and drum assembly 200 relative to each other and a fixed frame of reference. Actuators for performing these X-direction 214 translational movements may be coupled to one or more of the drum assembly 200, the track assembly 400, and the detection system 102 associated with the objective 104.

FIG. 2D shows a top view of the inner drum 201 and the objective 104. As shown, the inner drum 201 may move relative to the objective 104 in the X direction 214 and the Y direction 212, as discussed in relation to FIGS. 2B and 2C.

A combination of the relative movements between the inner drum 201 and the objective 104 shown in FIGS. 2B-2D may be performed by actuators of an actuation system 901 controlled by a control system 900 in order to scan the objective 104 across a plurality of locations over the curved substrate 202 on the inner drum 201 in order to image the sequencing events. Additional relative movement, for example X, Y, and/or Z rotational movements of the entire drum assembly 200 relative to the objective 104 may be performed by actuators of the actuation system 901 controlled by a control system 900 in order to precisely position, align and/or focus the objective 104 during imaging. The control system 900 may receive from any combination of input from one or more of position/acceleration/movement sensors of one or more components of the system 100, for example an encoder of actuator 203, and/or processed image data of the curved substrate 202 from the detection system 102, in order to control the relative movement of the objective 104 and inner drum 201.

FIGS. 3A-3D show examples of a portion 210 of an inner drum 201 and a substrate 203. As shown in FIG. 3A, the portion 210 of the inner drum 201 includes a recessed surface 215 between the ridges 209. One or more substrates 202 may be integrally formed with or coupled to the recessed surface 215. The recessed surface 215 may be cylindrical and one or more curved substrates 202 may wrap around the entire circumference of the recessed surface 215, or a portion thereof. For example, a single curved substrate 202 may wrap any amount from 1°-360° around the circumference of the inner drum 201. The substrate 203 may be formed for example of silicon or SiO₂. The substrate may be produced from a wafer, for example a silicon wafer or a SiO₂ wafer. The thickness of the wafer may be selected in order to be able to be flexed into the curved shape to match the inner drum radii without breaking. As shown in FIG. 3B, a curved substrate 202 may be planar prior to being coupled to the recessed surface 215. The recessed surface may have a circumference between about 25 mm and 25000 mm and a width between 1.0 mm and 30 mm. When coupled to the recessed surface 215 the curved substrate 202 may be bent in order to match the curvature of the recessed surface, as shown for example in FIG. 3C. As shown in FIG. 3D, a recessed surface 215 of a portion of an inner drum 201 may include multiple parallel curved substrates 202. Each of one or more substrates 202 on a recessed surface 215 may have a width of 25 mm to 500 mm.

Substrates 202 on the inner drum 201, for example as shown in FIGS. 3B and 3C, may be virtually and/or physically divided into an array of subregions during an imaging process. The curved substrate may define a patterned array of derivitized areas (“spots” or discrete spaced apart regions). The positions, or spots, may be organized as a regular, ordered array and are adapted to contain nucleic acid template molecules. In some approaches, the array includes more than 10⁵, more than 10⁶, more than 10⁷ sites, more than 10⁸ sites, more than 10⁹ sites, or more than 10¹⁰ sites, such as from 10⁵ to 10¹¹ sites or 10⁶ to 10¹⁰ sites. For example, the positions may be regions of the substrate surface derivatized to bind nucleic acid molecules (e.g., DNA nanoballs (DNBs), a template cluster produced by bridge amplification, or other templates), wells, or other structures. In some embodiments the surface of the substrate between spots is adapted to not bind nucleic acid molecules.

The control system may define one or more imaging paths on the curved substrate 202 within a control scheme for imaging the array of derivitized areas. The actuators of the actuation system are used to control the relative motion of the objective and substrates in order to image the substrates along the imaging paths. As shown for example in FIG. 3B, a substrate 202 may include a plurality of virtually defined imaging paths 217, indicated in the figures as the areas of the substrate between the dotted lines representing virtual boundaries between adjacent imaging paths. The curved substrate 202 may wrap entirely around the recessed surface 215 of the inner drum 201 and the imaging paths 217 may be circular rings around the inner drum 201. For example, FIG. 3C shows a representation of one circular ring imaging path 217, indicated with slashed lines, around the curved substrate 202 on the recessed surface 215.

The controller may cause the actuation system and detection system to sequentially scan the substrate along a plurality of ring imaging paths. To scan the plurality of ring imaging paths, the inner drum 201 may be rotated, for example at a constant speed, around the X-axis with the actuator 203. A constant rotation speed may result in a surface velocity of the substrate of 10 mm/sec to 200 mm/sec. With the actuation system, the drum assembly 200 and the objective 104 may be moved relative to each other in order to cause the field of view of the objective 104 to be positioned over a first ring imaging path. The width of each imaging path may correspond to the width of the FOV of the objective. The end of the objective 104 may be positioned by the actuation system within 20 microns of the curved substrate, within a precision of +/−0.05 microns. The detection system images the curved substrate 202 as the inner drum 201 makes a complete rotation in order to image an entire first ring imaging path. The drum assembly 200 and the objective 104 may then be moved by the actuation system in order to cause the field of view of the objective 104 to be positioned over a second ring imaging path and imaging of the second ring is performed over the course of an entire rotation of the inner drum 201, which may be rotating at the constant speed while imaging the first ring imaging path and the second ring imaging path, and while the FOV is moved between the first ring imaging path and the second ring imaging path. In examples, an objective may have a field of view 1.5 mm wide, and after each rotation of the inner drum the drum assembly may be translated in the X-direction by 1.5 mm, the width of the FOV, or less. For example, the translation distance may be less than the width of the FOV so that adjacent imaging paths overlap to ensure complete imaging of the entire substrate. The above steps for imaging an imaging path may be repeated for each imaging path on one or more curved substrates on the portion 210 of the inner drum 201. The actuation system may further be used to move the drum assembly 200 relative to the objective 104 so that the steps may be performed on the curved substrates on other portions 210 of the inner drum 201.

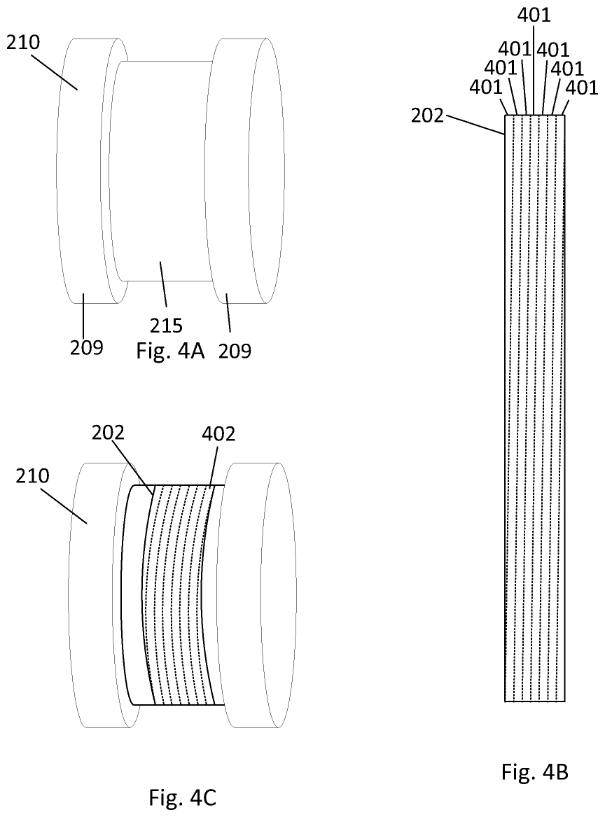

The control system may define imaging paths as spiral imaging paths, for example as shown in FIGS. 4A-4C. As shown in FIG. 4B, the control system may define on a curved substrate 202 a plurality of sub-paths 401 angled relative to an edge of the substrate 202 when viewed as a planar substrate so that when the substrate is curved around the inner drum the end of one sub-path 401 aligns with the beginning of another sub-path 401 in order to form a spiral imaging path 402. As shown, the spiral imaging path 402 may wind around a circumference of the inner drum a plurality of times. The control system may cause the actuation system and detection system to scan the substrate along the one or more spiral imaging paths on the curved substrate 202. To scan a spiral imaging path 402, the inner drum 201 may be rotated at a constant speed around the X-axis with the actuator 203. With the actuators of the actuation system, the drum assembly 200 and the objective 104 are moved relative to each other in order to cause the field of view of the objective 104 to be positioned at an end of a curved substrate where a spiral imaging path begins. Simultaneously with the inner drum rotating around the X-axis, the actuation system causes the drum assembly 200 to translate in the X-direction at a constant rate. The rate of rotation and translation may be coordinated so that the drum assembly 200 translates in the X-direction the width of the field of view of the objective 104, or less as discussed above to have overlap, during each rotation on the inner drum 201. In this way, an entire spiral imaging path, which may cover substantially all of a substrate, may be imaged in a single continuous imaging step wherein the rotation and translation are maintained at constant rates throughout the imaging of the spiral imaging path. These steps may be repeated for each imaging path on one or more curved substrates 202 on the portion 210 of the inner drum 201. The actuation system may further be used to move the drum assembly 200 relative to the objective 104 so that the steps for imaging spiral imaging paths may be performed on the curved substrates on other portions 210 of the inner drum 201.

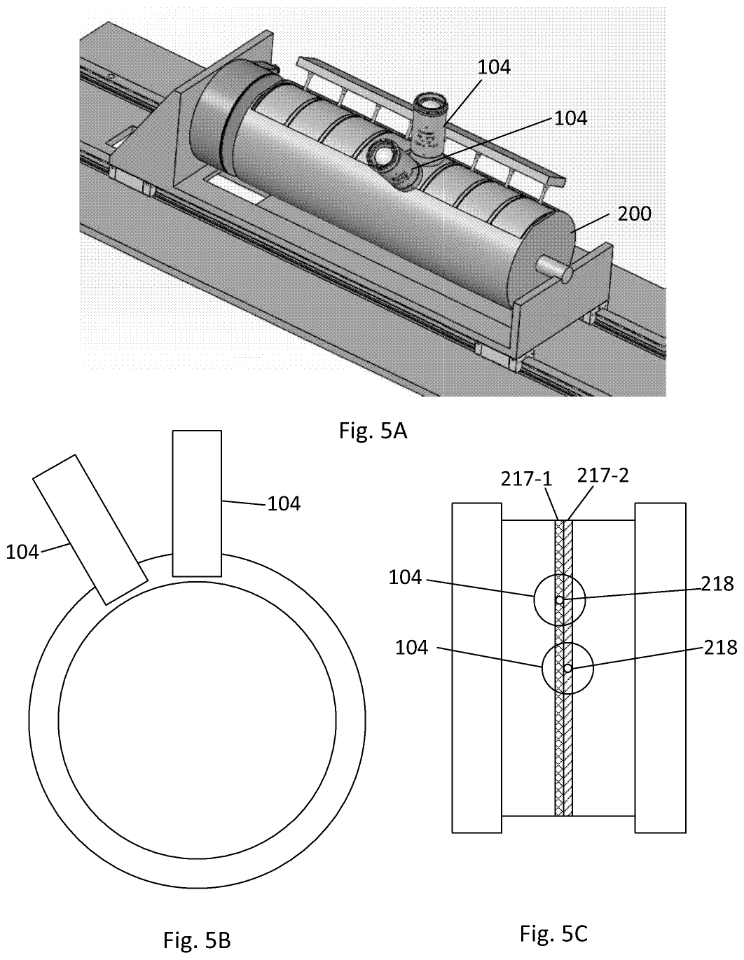

Utilizing the ring or spiral imaging paths with a continuously rotating inner drum 201 allows for increased imaging speed, and therefore an increased rate of generating sequencing data, compared to imagers which image a planar substrate by frequently stopping, slowing down, or changing the direction of the objective relative to the substrate between each transit of the objective relative to the substrate. The imaging speed may further be increased compared to planar substrate imaging systems by including two or more objectives, for example as shown in FIGS. 5A-5C. As shown in FIGS. 5A and 5B, two objectives 104 may be positioned at different radial positions around the drum assembly 200. Further, as shown in FIG. 5C, the fields of view 218 of the two objectives 104, which are much smaller than the profile of the objective, may be offset from one another in the X-direction. The offset in the X-direction may be substantially equal to the width of the field of views 218 so that the effective field of view of the detection system is twice as wide as a single objective detection system, thus doubling the imaging speed by imaging two imaging paths 217-1 217-2 simultaneously, wherein the adjacent imaging paths may be ring or spiral imaging paths and the rate of translation in the X-direction may be doubled. The actuation system may include actuators to separately cause relative movement for each of the two or more objectives relative to the drum assembly in order to separately position, align, and focus the different objectives.

As noted above, the one or more curved substrates may include nucleic acid template molecules (e.g., DNBs) immobilized at positions on the curved substrate. Prior to, during, and/or after imaging, reagents and wash buffers may be separately flowed through the flowcells defined by each chamber corresponding to each portion 210 of the inner drum 201. For example, as shown in FIG. 1B, the fluid delivery system 600 may comprise a plurality of delivery elements 601 for delivering reagents or other fluids, into each flowcell associated with each portion 210 of the inner drum 201. The delivery elements 601 may be positioned to deliver fluid onto a portion of the substrate on the inner drum prior to the portion passing under the objective to be imaged. The delivery element 601 may extend into the outer drum shell 204 through or proximate to the opening 216. Further, the outer drum shell 204 may include exit ports 602 at a bottom of each chamber, as shown for example in FIG. 6A. During imaging, and during chemistry steps that occur prior to and subsequent to the imaging step, the chamber may generally be an aqueous environment, which may be necessary to preserve the nucleic acid templates disposed therein on the curved substrate. The fluid delivery system 600 deliver fluids into the chamber so that a liquid surface 603 is maintained so that the tops of the recessed surfaces 215 are submerged in the aqueous environments. As shown for example in FIG. 6A, the objective 104 may be submerged below the liquid surface 603 during imaging of the substrate. In examples, the liquid surface 603 may be maintained below the top of the recessed surface 215 and surface tension of the liquid on the curved substrate may maintain an aqueous environment on the non-submerged portion of the substrate. The environment adjacent to the shell opening 216 may be controlled by an environment control system to have increased humidity in order to reduce and control evaporation of liquid within the outer drum shell 204. The shell opening 216, for example as shown in FIG. 6B, may include a coverslip 604 sealing the top of each chamber, and the objective may image the curved substrates through the coverslip 604.

In FIGS. 6A and 6B, the distance between the inner surface of the outer drum shell 204 and the recessed surface 215 may or may not be to scale. The distance between the inner surface of the outer drum shell 204 and the recessed surface 215 may be between 0.1 mm and 3.0 mm.

The reagents and wash buffers flowed through the chambers corresponding to each portion 210 of the inner drum 201, may drain out of the exit ports 602 and be disposed of, or may be flowed to a recycling system 605, as shown for example in FIGS. 6A and 6B. The recycling system 605 may separately store fluids drawings from exit ports 602 to be reused in subsequent processes. For example, the previously used reagents may be stored and used in subsequent processes in order to provide the benefit reducing the total amount of reagents used.

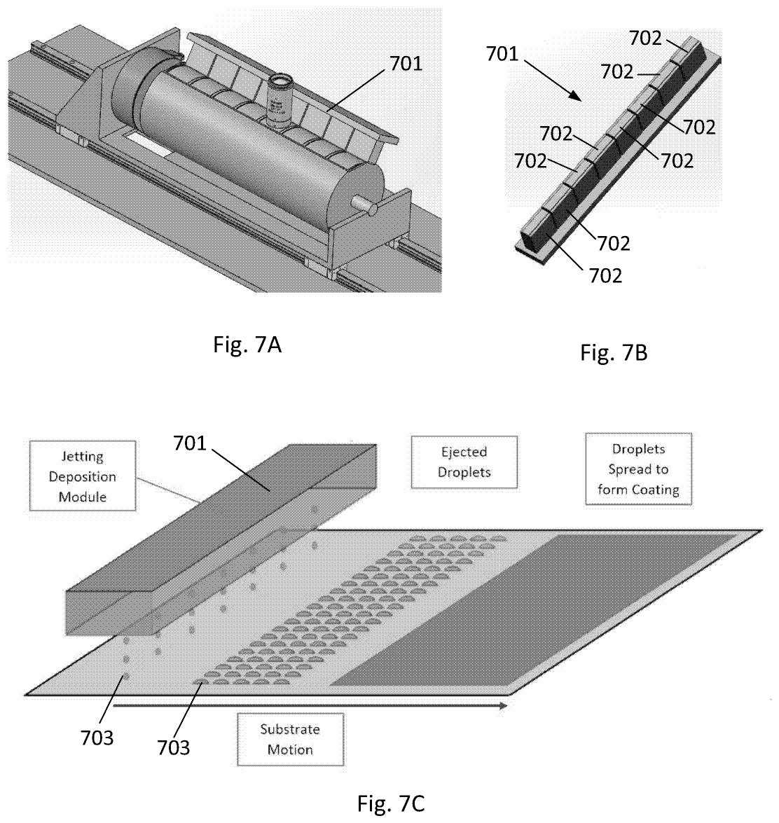

As shown in FIGS. 6A and 6B, the fluid delivery system 600 may use the delivery elements 601 to fill the chambers with reagents and wash buffers. The fluid delivery system may include a temperature control system as part of the environment control system, which may include heaters, coolers, and/or temperature sensors, in order to deliver fluids at a target temperature in order to promote sequencing reactions caused by the reagents. In examples, the chamber may not be filed with reagents, and instead reagents may be jetted in droplets onto the curved substrates 202. For example, as shown in FIG. 7A, additionally or alternatively to the delivery element 601 shown in FIG. 1B, an optical imaging system may include a jetting print head 701 for each chamber. The jetting print head 701, for example as shown in FIG. 7B may include a plurality of subheads 702 in a row. The width of each subhead 702 of the jetting print head 701 may correspond to the width between the ridges 209, so that in a single rotation of the inner drum 201, the entire recessed surface 215 including one or more curved substrates 202 may have reagent jetted onto it.

As shown in FIG. 7C, the jetting print head 701 may jet droplets 703 of reagent or wash buffer in rows on the curved substrate 202. As the curved substrate 202 is rotated with the inner drum 201, an array of droplets may be formed. The amount of fluid per droplet and the surface tension of the droplet to the curved substrate may be selected so that the droplets spread to form an even coating, for example as shown in FIG. 7C. The layer of spread droplets may be 0.5 microns thick. Alternatively, individual droplets may be jetted onto each of the derivatized areas at which a template is immobilized on the curved substrate 202, and may not spread into the underivitized or differently derivatized surface between binding sites to form an even coating over the surface. For example, e.g., one or more individual droplets may be disposed at each site occupied by a DNB).

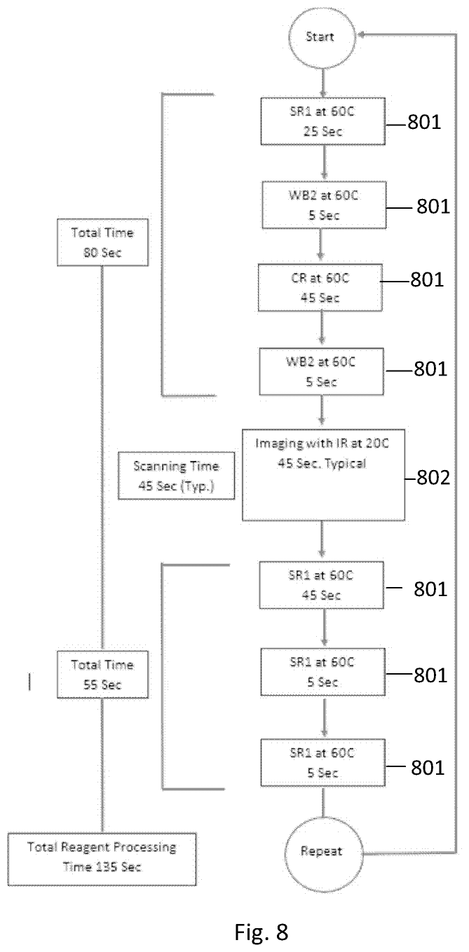

The number of chambers defining the flowcells of a drum assembly may correspond to the number of distinct chemistry and imaging steps in a sequencing process, for example the steps of a sequencing process to read one base. For example, as shown in FIG. 8, a sequencing process may include 7 reagent/wash buffer steps 801, and one imaging step 802, and the corresponding drum assembly may include 8 chambers, one chamber for each step. At any time, each of the chambers may then be used to perform a different step in the sequencing process. In embodiments, two or more steps of a sequencing process may occur in one chamber while one or more steps of the sequencing process are occurring simultaneously in another chamber, and the number of chambers defining the flowcells of a drum may be less than the total number of chemistry and imaging steps of the sequencing process. Once each chamber has performed the respective process step(s), each chamber may then be shifted to the respective subsequent process step(s). For example, once an imaging step is performed in a first chamber, the drum assembly 200 and objective 104 may be shifted by the actuation system in the X-direction so that the objective may perform the imaging step on a second chamber, and the reagent delivery system may perform a non-imaging chemistry step on the first chamber. In other words, the sequencing process flow, for example as shown in FIG. 8, may be performed in parallel simultaneously for each chamber, wherein each chamber is on a different step of the sequencing process flow. This arrangement of multiple chambers in a drum assembly may be advantageous including for reasons described in U.S. Pat. No. 10,351,909 B2 (“DNA sequencing from high density DNA arrays using asynchronous reactions”), which is incorporated by reference herein in its entirety.

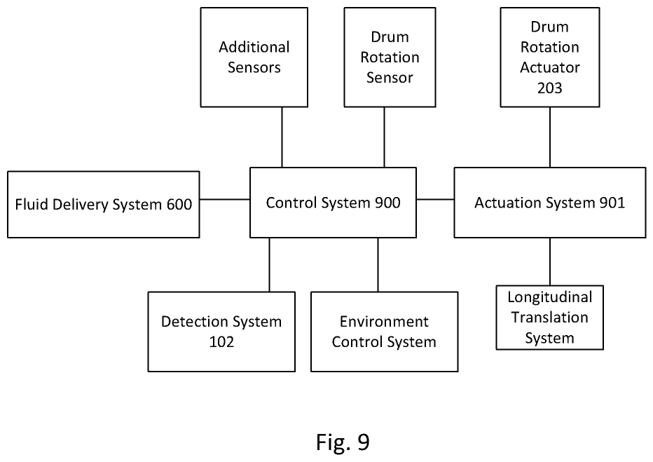

FIG. 9 shows a schematic of the sub-systems of a sequencing system. As shown, a control system may be coupled to send and receive signals to each of the components of the system in order to control the system, as described above.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

While the foregoing disclosure shows illustrative aspects of the disclosure, it should be noted that various changes and modifications could be made herein without departing from the scope of the disclosure as defined by the appended claims. The functions, steps and/or actions of the method claims in accordance with the aspects of the disclosure described herein need not be performed in any particular order. Furthermore, although elements of the disclosure may be described or claimed in the singular, the plural is contemplated unless limitation to the singular is explicitly stated.

1. A nucleic acid sequencing system, the system comprising:
a drum defining an outer surface and a longitudinal axis;
a non-planar substrate coupled to the outer surface of the drum and configured to support a plurality of nucleic acid samples;
an actuation system configured to rotate the drum around the longitudinal axis; and
a detection system configured to detect sequencing events on the non-planar substrate while the non-planar substrate is rotated relative to the detection system around the longitudinal axis by the actuation system.

2. The nucleic acid sequencing system of claim 1, wherein the outer surface is cylindrical.

3. The nucleic acid sequencing system of claim 2, wherein the non-planar substrate is curved around the outer surface of the drum.

4. The nucleic acid sequencing system of claim 2, wherein the actuation system is further configured to translate the drum along the longitudinal axis, and
wherein the detection system is further configured to detect sequencing events on the non-planar substrate while the non-planar substrate is translated relative to the detection system along the longitudinal axis by the actuation system.

5. The nucleic acid sequencing system of claim 1, wherein the detection system is an optical detection system comprising at least one objective.

6. The nucleic acid sequencing system of claim 5, wherein the at least one objective comprises two objective configured to image portions of the substrate offset radially and longitudinally of the longitudinal axis of the drum.

7. The nucleic acid sequencing system of claim 5, further comprising:
a drum assembly comprising:
the drum; and
an outer drum shell defining an interior cavity, wherein the inner drum is positioned within the inner cavity, and wherein the actuation system is configured to rotate the inner drum within the inner cavity of the outer drum shell.

8. The nucleic acid sequencing system of claim 7, further comprising:
a track assembly coupled to the drum assembly,
wherein the actuation system is configured translate the drum assembly in a direction parallel to the longitudinal axis in order for the at least one objective to image different portions of the curved substrate in a direction parallel to the longitudinal axis as the inner drum is rotating around the longitudinal axis.

9. The nucleic acid sequencing system of claim 8, further comprising a control system, wherein the control system is configured to control the actuation system in order to rotate the inner drum and translate the inner drum in order for the objective to image a predefined imaging path on the curved substrate.

10. The nucleic acid sequencing system of claim 9, wherein the predefined imaging path is a ring around a circumference of the inner drum.

11. The nucleic acid sequencing system of claim 9, wherein the predefined imaging path is a spiral winding around the inner drum a plurality of times.

12. The nucleic acid sequencing system of claim 7, wherein the drum comprises a plurality of ridges, and a plurality of recessed surface between adjacent ridges of the plurality of ridges comprising a first recessed surface,
wherein non-planar substrate is coupled to the first recessed surface.

13. The nucleic acid sequencing system of claim 7, further comprising:
a fluid delivery system configured to deliver fluid to the interior cavity of the outer drum shell in order to perform a sequencing process on the non-planar substrate.

14. The nucleic acid sequencing system of claim 13, wherein the fluid delivery system comprises a jetting print head configured to jet droplets of a reagent onto the non-planar substrate.

15. The nucleic acid sequencing system of claim 13, wherein the outer drum shell comprises an exit port configured to drain fluid within the interior cavity delivered by the fluid delivery system.

16. The nucleic acid sequencing system of claim 15, wherein the fluid delivery system comprises a recycling system for capturing fluid drained from the exit port in order to reuse the fluid.

17. The nucleic acid sequencing system of claim 1, wherein the non-planar substrate comprises an ordered array of discrete spaced apart regions (“spots”),
wherein said discrete spaced apart regions are adapted to immobilize nucleic acids.

18. The nucleic acid sequencing system of claim 17, further comprising:
nucleic acids immobilized on the discrete spaced apart regions of the array.

19. The nucleic acid sequencing system of claim 18, wherein the nucleic acids immobilized on the discrete spaced apart regions are DNBs or PCR products.

20. A method of nucleic acid sequencing, the method comprising:
rotating a drum defining an outer surface around a longitudinal axis of the drum with an actuation system; and
detecting sequencing events, with a detection system, on a non-planar substrate coupled to the drum while the non-planar substrate is rotated relative to the detection system around the longitudinal axis by the actuation system.

21. The method of claim 20, wherein detecting sequencing events is performed while the drum is rotated at a constant speed.

22. The method of claim 20, wherein detecting sequencing events on the non-planar substrate comprises:
positioning an objective of the detection system at a first longitudinal position relative to the longitudinal axis of the drum;
maintaining the objective at the first longitudinal position as the drum is rotated relative to the detection system around the longitudinal axis by the actuation system at least one full rotation in order to image a first portion of the non-planar substrate around a first ring imaging path;
positioning the objective at a second longitudinal position relative to the longitudinal axis of the drum; and
maintaining the objective at the second longitudinal position as the drum is rotated relative to the detection system around the longitudinal axis by the actuation system at least one full rotation in order to image a second portion of the non-planar substrate, different than the first portion, around a second ring imaging path.

23. The method of claim 20, wherein detecting sequencing events on the non-planar substrate comprises:
positioning an objective of the detection system at a first longitudinal position relative to the longitudinal axis of the drum; and
translating the objective at a constant speed from the first longitudinal position to a second longitudinal position as the drum is rotated relative to the detection system around the longitudinal axis by the actuation system in order to image a spiral imaging path around the non-planar substrate.
